(19) United States
(12) Patent Application Publication
(10) Pub. No.: US 2024/0241127 A1
(43) Pub. Date: Jul. 18, 2024

(54) METHOD FOR DETERMINING PROSTATE CANCER

(71) Applicant: HAMAMATSU PHOTONICS K.K., Hamamatsu-shi (JP)

(72) Inventors: Hiroyuki MATSUMOTO, Hamamatsu-shi (JP); Tomomi SHINKE, Hamamatsu-shi (JP); Hiroyuki OKADA, Hamamatsu-shi (JP)

(73) Assignee: HAMAMATSU PHOTONICS K.K., Hamamatsu-shi (JP)

(21) Appl. No.: 18/389,287

(22) Filed: Nov. 14, 2023

(30) Foreign Application Priority Data
Jan. 18, 2023 (JP) 2023-006009

(51) Int. Cl. G01N 33/574; G01N 33/70

(52) U.S. Cl. CPC G01N 33/57434; G01N 33/57488; G01N 33/70; G01N 2333/811

(57) ABSTRACT

There is provide a method for determining whether a subject has developed or is suspected to have developed prostate cancer is provided, the method including: comparing a prostate-specific antigen (PSA) level in serum with a first threshold value; comparing an α1-antitrypsin (AAT) level in urine with a second threshold value; and determining whether a subject in which the PSA level in serum is the first threshold value or more and the AAT level in urine is the second threshold value or more has developed or is suspected to have developed prostate cancer.

### CROSS-REFERENCE TO RELATED APPLICATION

This application claims the benefit of Japanese patent application No. 2023-006009 filed on Jan. 18, 2023, the disclosure of which is herein incorporated by reference in its entirety.

### TECHNICAL FIELD

The present invention relates to a method for determining prostate cancer which is for determining whether a subject has developed or is suspected to have developed prostate cancer.

### BACKGROUND

The incidence rate and the mortality rate of prostate cancer are increasing year by year. Conventionally, a PSA test, which is an examination for quantitatively measuring prostate-specific antigen (PSA) in serum, has mainly been performed as an examination for prostate cancer. The PSA test is a method for determining whether a subject in which a PSA level in serum is equal to or more than a predetermined threshold value (usually equal to or more than 4 ng/ml) has prostate cancer or is suspected to have prostate cancer (for example, Haese A. et al., “Clinical Evaluation of the Elecsys Total Prostate-specific Antigen Assay on the Elecsys 1010 and 2010 Systems”, Clinical Chemistry 48(6), 944-947 (2002)).

In addition, a method for examining for cancer using the urine of a subject is also known. For example, Japanese Unexamined Patent Publication No. 2015-169608 discloses a method for examining for cancer, the method including: a pretreatment step of subjecting urine collected from a subject to a predetermined pretreatment to obtain treated urine; a quantitative measurement step of quantitatively measuring a level of α1-antitrypsin with a molecular weight of 52 KDa in the treated urine by enzyme-linked immunosorbent assay (ELISA); and a determination step of determining that the subject has developed cancer or may have developed cancer when the obtained quantitative value exceeds a preliminarily set threshold value.

### SUMMARY

As disclosed in Haese A. et al., “Clinical Evaluation of the Elecsys Total Prostate-specific Antigen Assay on the Elecsys 1010 and 2010 Systems”, Clinical Chemistry 48(6), 944-947 (2002), the PSA test has high sensitivity for prostate cancer but has low specificity therefor. In addition, according to the studies of the inventors of the present invention, it was clarified that sensitivity was high but specificity was low also in a method for determining prostate cancer based on an α1-antitrypsin (hereinafter also referred to as “AAT”) level in urine (refer to No. 8 in Examples to be described later). In all of these examinations, since specificity was low, there was a problem of many false positives.

An object of the present invention is to provide a method for determining prostate cancer with improved specificity while maintaining sensitivity.

The inventors of the present invention have unexpectedly found that, by combining the determination based on a PSA level in serum and the determination based on an AAT level in urine, a method for determining prostate cancer in which sensitivity and specificity are both high is achieved. In general, even when examinations with high sensitivity and low specificity are combined, it cannot be said that the specificity is improved. The present invention is based on this new finding.

The present invention relates to a method for determining whether a subject has developed or is suspected to have developed prostate cancer (hereinafter also referred to as “determination method”), the method including: comparing a prostate-specific antigen (PSA) level in serum with a first threshold value; comparing an α1-antitrypsin (AAT) level in urine with a second threshold value; and determining whether a subject in which the PSA level in serum is the first threshold value or more and the AAT level in urine is the second threshold value or more has developed or is suspected to have developed prostate cancer.

In the determination method of the present invention, since the determination based on the PSA level in serum and the determination based on the AAT level in urine are combined, both sensitivity and specificity are high.

In the above-mentioned determination method, the AAT level in urine is preferably a level standardized with an internal standard substance. By using the level standardized with an internal standard substance as the AAT level in urine, more accurate determination can be performed. Furthermore, the internal standard substance is preferably creatinine (Cre) in urine.

In the above-mentioned determination method, when the level standardized by creatinine (Cre) in urine is used as the AAT level in urine, the second threshold value may be in a range of equal to or more than 0.01 μg/mg and less than 2 μg/mg.

In the above-mentioned determination method, the first threshold value may be in a range of equal to or more than 1 ng/mL and equal to or less than 5 ng/mL.

The present invention can also be regarded as a screening method for selecting a subject that has developed or is suspected to have developed prostate cancer, the method including: comparing a prostate-specific antigen (PSA) level in serum with a first threshold value; comparing an α1-antitrypsin (AAT) level in urine with a second threshold value; and selecting a subject in which the PSA level in serum is the first threshold value or more and the AAT level in urine is the second threshold value or more as a subject that has developed or is suspected to have developed prostate cancer.

The present invention can also be regarded as a data collection method for determining whether a subject has developed or is suspected to have developed prostate cancer, the method including: comparing a prostate-specific antigen (PSA) level in serum with a first threshold value; and comparing an α1-antitrypsin (AAT) level in urine with a second threshold value.

The present invention includes each of the following inventions, for example.

[1]

A method for determining whether a subject has developed or is suspected to have developed prostate cancer, the method including:
comparing a prostate-specific antigen (PSA) level in serum with a first threshold value;
comparing an α1-antitrypsin (AAT) level in urine with a second threshold value; and
determining whether a subject in which the PSA level in serum is the first threshold value or more and the AAT level in urine is the second threshold value or more has developed or is suspected to have developed prostate cancer.

[2]

A screening method for selecting a subject that has developed or is suspected to have developed prostate cancer, the method including:
comparing a prostate-specific antigen (PSA) level in serum with a first threshold value;
comparing an α1-antitrypsin (AAT) level in urine with a second threshold value; and
selecting a subject in which the PSA level in serum is the first threshold value or more and the AAT level in urine is the second threshold value or more as a subject that has developed or is suspected to have developed prostate cancer.

[3]

A data collection method for determining whether a subject has developed or is suspected to have developed prostate cancer, the method including:
comparing a prostate-specific antigen (PSA) level in serum with a first threshold value; and
comparing an α1-antitrypsin (AAT) level in urine with a second threshold value.

[4]

The method according to any one of [1] to [3], in which the AAT level in urine is a level standardized with an internal standard substance.

[5]

The method according to [4], in which the internal standard substance is creatinine (Cre) in urine.

[6]

The method according to [5], in which the AAT level in urine is a level standardized by dividing the AAT level in urine by a Cre level in urine.

[7]

The method according to [6], in which the second threshold value is in a range of equal to or more than 0.01 μg/mg to less than 2 μg/mg.

[8]

The method according to any one of [1] to [7], in which the first threshold value is in a range of equal to or more than 1 ng/mL and equal to or less than 5 ng/mL.

According to the present invention, a method for determining prostate cancer with improved specificity while maintaining sensitivity can be provided.

### BRIEF DESCRIPTION OF THE DRAWINGS

FIG. 1 is a scatter diagram plotting PSA levels in serum and AAT/Cre values in urine.

FIG. 2 is a diagram in which auxiliary lines indicating set threshold values were added to the scatter diagram of FIG. 1 and the scale was enlarged.

### DETAILED DESCRIPTION

Embodiments for carrying out the present invention will be described in detail below. However, the present invention is not limited to the following embodiments.

### [Determination Method]

A determination method of the present embodiment is a method for determining whether a subject has developed or is suspected to have developed prostate cancer, the method including: comparing a prostate-specific antigen (PSA) level in serum with a first threshold value; comparing an α1-antitrypsin (AAT) level in urine with a second threshold value; and determining whether a subject in which the PSA level in serum is the first threshold value or more and the AAT level in urine is the second threshold value or more has developed or is suspected to have developed prostate cancer.

For example, the subject may be humans, monkeys, mice, and rats, but is preferably humans.

Comparing the PSA level in serum with the first threshold value may be determining whether the PSA level in serum is equal to or more than the first threshold value or less than the first threshold value.

For example, the first threshold value may be set by a statistical treatment using serum obtained from the subject, or may be set based on a ROC curve.

In the setting by the statistical treatment, for example, serum obtained from a subject who has been preliminarily diagnosed whether the subject has developed prostate cancer or not is used as a sample to obtain data on the PSA level in the serum of the subject. By the statistical treatment of data on “whether or not prostate cancer developed” obtained from a plurality of subjects (50 subjects, for example) and data on the “PSA level in serum,” the correlation between these data is analyzed. The threshold value may be set based on the results of the analysis such that the specificity is high while maintaining the sensitivity, and among these, for example, the threshold value can be set according to purposes such as whether to emphasize a high level of a positive rate (high sensitivity), whether to emphasize a low level of a false positive rate (high specificity), and to what extent a positive rate and a false positive rate are balanced.

As a method for setting the threshold value based on a ROC curve, for example, a point with the smallest distance from an upper left corner may be used as the threshold value, a point (Youden Index) farthest from an oblique dotted line from which the area under the ROC curve is 0.500 may be used as the threshold value, or a threshold value may be set such that sensitivity or specificity is arbitrary.

Although the first threshold value is not particularly limited, for example, it may be in a range of 1 ng/mL or more and 5 ng/ml or less, or may be in a range of 1.5 ng/mL or more and 4.5 ng/ml or less, 2 ng/ml or more and 4 ng/ml or less, or 3 ng/ml or more and 4 ng/ml or less.

The determination method according to one embodiment may further include quantitatively measuring the PSA level in serum. The PSA level in serum can be quantitatively measured from serum by methods commonly used by those skilled in the art to quantitatively measure or identify proteins. The serum can be obtained by conventional methods such as a method using a blood separation filter on blood collected from a subject or a method of centrifuging blood collected from a subject.

A method for quantitatively measuring the PSA level in serum is not particularly limited as long as the PSA level in serum can be quantitatively measured, and examples thereof include antigen-antibody reaction detection methods such as western blotting, enzyme-linked immunosorbent assay (ELISA), flow strip assay, and latex agglutination immunoassay; amino acid sequencing; and mass spectrometry.

Comparing the AAT level in urine with the second threshold value may be determining whether the AAT level in urine is equal to or more than the second threshold value or less than the second threshold value.

AAT in urine may be any one as long as it can be detected in urine, and may be full-length AAT alone or may be full-length AAT and partially hydrolyzed AAT.

The AAT level in urine may be a level standardized with an internal standard substance. Thus, the AAT level in urine can be quantitatively measured more accurately, which further makes it possible to perform more accurate determination. The internal standard substance is preferably a substance present at a constant concentration in urine. By standardizing the AAT level with the internal standard substance, for example, a value without the influence of the amount of urine can be obtained. Standardization may be performed by dividing the AAT level in urine by the internal standard substance level in urine, for example.

Examples of the internal standard substance include total protein, albumin, and creatinine (Cre), among which Cre is preferable.

Setting of the second threshold value can be set in the same manner as the above-mentioned setting of the first threshold value.

The second threshold value is not particularly limited, but may be in a range of 0.01 μg/mg or more, 0.03 μg/mg or more, 0.05 μg/mg or more, 0.1 μg/mg or more, 0.2 μg/mg or more, 0.3 μg/mg or more, 0.4 μg/mg or more, 0.5 μg/mg or more, 0.6 μg/mg or more, 0.7 μg/mg or more, 0.8 μg/mg or more, 0.9 μg/mg or more, or 1 μg/mg or more, for example. In addition, the second threshold value may be in a range of 9 μg/mg or less, 8 μg/mg or less, 7 μg/mg or less, 6 μg/mg or less, 5 μg/mg or less, 4 μg/mg or less, 3 μg/mg or less, 2 μg/mg or less, or 1.3 μg/mg or less, for example.

When the AAT level in urine is standardized by dividing the AAT level in urine by Cre in urine, the second threshold value may be in a range of equal to or more than 0.01 μg/mg and less than 2 μg/mg, or may be in a range of 0.03 μg/mg or more and 1.8 μg/mg or less, 0.1 μg/mg or more and 1.5 μg/mg or less, 0.5 μg/mg or more and 1.3 μg/mg or less, or 0.8 μg/mg or more and 1.3 μg/mg or less from the viewpoint of improving specificity while further maintaining sensitivity.

The determination method according to one embodiment may further include quantitatively measuring the AAT level in urine. The AAT level in urine can be quantitatively measured from urine by methods commonly used by those skilled in the art to quantitatively measure or identify proteins.

A method for quantitatively measuring the AAT level in urine is not particularly limited as long as the AAT level in urine can be quantitatively measured, and examples thereof include antigen-antibody reaction detection methods such as western blotting, ELISA, flow strip assay, and latex agglutination immunoassay; amino acid sequencing; and mass spectrometry, among which the antigen-antibody reaction detection methods are preferable, and ELISA is more preferable.

When the AAT level in urine is quantitatively measured by ELISA, ELISA may be any one as long as the AAT level in urine is quantitatively measured by utilizing the property that AAT binds to an anti-α1-antitrypsin antibody or a functional fragment thereof. For example, ELISA may be any of a direct adsorption method, a sandwich method, or a competitive method. ELISA is a well-known technique in the present technical field, and those skilled in the art can appropriately set various conditions (for example, type of reagent, temperature, time, number of times, and the like) for selection of reagents such as antibodies and labels, blocking, antigen-antibody reaction, and various washings; and various conditions such as label detection conditions.

When quantitatively measuring the AAT level in urine, the obtained urine may be used as it is for the quantitative measurement, or the obtained urine that has been pretreated may be used for quantitative measurement. A pretreatment may be appropriately selected depending on quantitative measurement methods, and examples thereof include a treatment to remove contaminants other than AAT in urine (hereinafter also referred to as “removal treatment”), and a treatment to denature AAT in urine (hereinafter also referred to as “denaturation treatment”). By performing the pretreatment, the AAT level in urine can be quantitatively measured more accurately.

Examples of the removal treatment include a method by gel filtration, a method in which electrophoresis and excision from a gel are combined, a method by size exclusion chromatography, a method by ultrafiltration, a method by reverse osmosis, a method by centrifugation, a method by precipitation, and a method using dialysis. For the removal treatment, one type may be performed alone, or two or more types may be combined and performed.

In the present specification, the term “denaturation treatment” means a treatment for denaturing a protein by disrupting the highly ordered structure of the protein. Examples of the denaturation treatment include a heat denaturation treatment, a low temperature denaturation treatment, an acid denaturation treatment, an alkali denaturation treatment, a pressure denaturation treatment, an ultrasonic treatment, a denaturation treatment with a denaturant, and an organic solvent treatment. For the denaturation treatment, one type may be performed alone, or two or more types may be combined and performed.

Examples of the denaturant include denaturants that disrupt hydrogen bonds, such as urea and a hydrochloride salt of guanidine (or guanidine hydrochloride); denaturants that disrupt hydrophobic bonds, such as sodium dodecyl sulfate (SDS), hexadecyltrimethylammonium bromide (CTAB), sodium cholate; and denaturants that reduce disulfide bonds, such as 2-mercaptoethanol and dithiothreitol. For the denaturant, one type may be used alone, or two or more types may be used in combination.

Comparing the PSA level in serum to the first threshold value and comparing the AAT level in urine to the second threshold value can be performed in any order. Furthermore, for example, comparing the AAT level in urine to the second threshold value may be performed only when the PSA level in serum is equal to or more than the first threshold value as a result of comparing the PSA level in serum to the first threshold value. Furthermore, comparing the PSA level in serum to the first threshold value may be performed only when the AAT level in urine is equal to or more than the second threshold value as a result of comparing the AAT level in urine to the second threshold value. Furthermore, for example, for only subjects, in which the PSA level in serum is equal to or more than the first threshold value and the AAT level in urine is equal to or less than the second threshold value at a certain first time point, the AAT level in urine may be compared to the second threshold value at the second time point after a certain period of time has elapsed from the first time point. Conversely, for only subjects, in which the PSA level in serum is equal to or less than the first threshold value and the AAT level in urine is equal to or more than the second threshold value at a certain first time point, the PSA level in serum may be compared to the first threshold value at the second time point after a certain period of time has elapsed from the first time point.

Determining that a subject, in which the PSA level in serum is equal to or more than the first threshold value and the AAT level in urine is equal to or more than the second threshold value, has developed or is suspected to have developed prostate cancer is based on comparing the PSA level in serum to the first threshold value and comparing the AAT level in urine to the second threshold value, and thus whether the subject has developed or is suspected to have developed prostate cancer can be determined mechanically.

### [Screening Method]

A screening method according to the present embodiment is a screening method for selecting a subject that has developed or is suspected to have developed prostate cancer, the method including: comparing a prostate-specific antigen (PSA) level in serum with a first threshold value; comparing an α1-antitrypsin (AAT) level in urine with a second threshold value; and selecting a subject in which the PSA level in serum is the first threshold value or more and the AAT level in urine is the second threshold value or more as a subject that has developed or is suspected to have developed prostate cancer.

Comparing the prostate-specific antigen (PSA) level in serum with the first threshold value and comparing the α1-antitrypsin (AAT) level in urine with the second threshold value in the screening method according to the present embodiment are as described above. Selecting a subject, in which the PSA level in serum is equal to or more than the first threshold value and the AAT level in urine is equal to or more than the second threshold value, as a subject that has developed or is suspected to have developed prostate cancer is the same as the above-mentioned determination that the subject, in which the PSA level in serum is equal to or more than the first threshold value and the AAT level in urine is equal to or more than the second threshold value, has developed or is suspected to have developed prostate cancer.

The screening method according to the present embodiment can apply each of the embodiments described in the above-mentioned determination method.

### [Data Collection Method]

A data collection method according to the present embodiment is a data collection method for determining whether a subject has developed or is suspected to have developed prostate cancer, the method including: comparing a prostate-specific antigen (PSA) level in serum with a first threshold value; and comparing an α1-antitrypsin (AAT) level in urine with a second threshold value.

Comparing the prostate-specific antigen (PSA) level in serum with the first threshold value and comparing the α1-antitrypsin (AAT) level in urine with the second threshold value in the data collection method according to the present embodiment are as described above. In addition, comparative data 1 can be obtained by comparing the prostate-specific antigen (PSA) level in serum to the first threshold value. Similarly, comparative data 2 can be obtained by comparing the α1-antitrypsin (AAT) level in urine to the second threshold value. The data collection method according to the present embodiment can also be regarded as a method for collecting the comparative data 1 and the comparative data 2.

The data collection method according to the present embodiment can apply each of the embodiments described in the above-mentioned determination method. In addition, the data collection method according to the present embodiment may include: collecting data of the PSA level in serum obtained by quantitatively measuring the PSA level in serum; collecting data of the AAT level in urine obtained by quantitatively measuring the AAT level in urine; and collecting data of the standardized AAT level in urine (for example, a AAT/Cre value in urine) obtained by standardizing the AAT level in urine with an internal standard substance.

### Examples

Hereinbelow, the present invention will be described more specifically based on examples. However, the present invention is not limited to these.

### (1. Quantitative Measurement of PSA Level in Serum and AAT Level in Urine)

On the day of the checkup, urine and blood were collected from 718 men undergoing cancer screening in the state before various examinations. Using the collected urine, the α1-antitrypsin level and the creatinine level in the urine were quantitatively measured. Subsequently, the value obtained by dividing the α1-antitrypsin level (unit: μg/mL) in urine by the creatinine level (unit: mg/mL) in urine (hereinafter also referred to as “AAT/Cre value (unit: μg/mg) in urine”) was obtained as follows. Using the collected blood, the PSA level (unit: ng/mL) in serum was quantitatively measured as follows.

### <Quantitative Measurement of AAT Level in Urine>

The urine collected from the subjects was centrifuged (3,000×g, 4° C., 10 minutes). A 50 μl portion of the obtained supernatant was taken, mixed with an equal volume of 25 mM Tris-HCl buffer (pH 6.8), and then subjected to ultrafiltration to remove a fraction in which solutes with a molecular weight of 10 kDa or less were fractionated. The ultrafiltration is performed by adding onto a membrane of an ultrafiltration device (manufactured by Millipore, Microcon 10) to centrifuge (13,200×g, 4° C., 60 minutes), and adding 100 μl of 25 mM Tris-HCl buffer (pH 6.8) onto the membrane after centrifugation to further repeat the centrifuging operation step (13,200×g, 4° C., 60 minutes) four times in total. Finally, the membrane of the ultrafiltration device was reversed, and 50 μl of 25 mM Tris-HCl buffer (pH 6.8) was added onto the membrane to perform the centrifugation operation (13,200×g, 4° C., 10 minutes). The obtained solution was used as a measurement sample and provided for quantitative measurement of the AAT level in urine by ELISA.

A system for quantitatively measuring the AAT level in urine was constructed by the following procedure. An anti-α1-antitrypsin polyclonal antibody (manufactured by Bethyl Laboratories, Inc.) was dissolved in 0.1 M carbonate buffer (pH 9.5) such that the concentration was 125 ng/100 μl, and 100 μl thereof was added to each well of an Immuno plate (MaxiSorp) manufactured by Nunc to cause a reaction (immobilization reaction) at 4° C. for one day. After the immobilization reaction, each well was washed three times with Dulbecco's phosphate buffered saline (hereinafter “D-PBS(−)”), and 200 μl of a polymer-based blocking solution (N101, manufactured by NOF CORPORATION) diluted 5-fold with ultrapure water was added to each well to cause a reaction (blocking reaction) at 4° C. for one day. After the blocking reaction, each well was washed five times with D-PBS(−) containing 0.05% Tween 20 to obtain an anti-α1-antitrypsin antibody-immobilized plate.

D-PBS(−) containing 0.1% bovine serum albumin and 0.05% Tween 20 was added to the above-mentioned measurement sample to prepare dilution series of various concentrations, and using the above-mentioned anti-α1-antitrypsin antibody-immobilized plate, measurement was performed with a detection system by ELISA. Using two wells for the same concentration, 100 μl was added to each well (duplicate addition) to cause a reaction at 4° C. for one day. After the reaction, each well was washed five times with D-PBS(−) containing 0.05% Tween 20, and 100 μl of an HRP-labeled anti-α1-antitrypsin polyclonal antibody (Bethyl Laboratories, Inc.) prepared at 10 ng/100 μl with D-PBS(−) containing 0.1% bovine serum albumin and 0.05% Tween 20 was added to each well to cause a reaction for 1 hour at room temperature while shielding light. After the reaction, each well was washed five times with D-PBS(−) containing 0.05% Tween 20. 100 μl of a TMB solution (manufactured by Sigma-Aldrich) was added to each well to cause a reaction for 15 minutes at room temperature while shielding light. After the reaction, 100 μl of 1 N sulfuric acid was added to each well to cause a reaction at room temperature for 15 minutes. After the reaction, an absorbance at 450 nm was measured using a plate reader (manufactured by Molecular Devices, LLC, SPECTRAMAX 250).

### <Quantitative Measurement of Creatinine Level in Urine>

The urine collected from the subjects was centrifuged (3,000×g, 4° ° C., 10 minutes). The obtained supernatant was used to quantitatively measure the creatinine level using an automated biochemistry analyzer JCA-BM 6050 BioMajesty (manufactured by JEOL Ltd.) and using a creatinine kit CICA Liquid-S CRE (manufactured by Kanto Chemical Co., Inc.).

From the obtained quantitative value and from a calibration curve of a human AAT standard protein obtained by simultaneous measurement in separate wells on the same plate, the quantitative value of the AAT level in urine was obtained and then divided by the quantitative value of the creatinine level in the same sample to calculate the AAT/Cre value (unit: μg/mg) in urine.

### <Quantitative Measurement of PSA Level in Serum>

The blood collected from the subjects using a blood collection tube not containing an anticoagulant was centrifuged (3,500 rpm, 4° C., 5 minutes) to obtain serum. The obtained serum was used to quantitatively measure the PSA level using UniCel DxI 800 Access Immunoassay System (manufactured by Beckman Coulter, Inc.) and using a prostate-specific antigen kit Access Hybritech PSA (manufactured by Beckman Coulter, Inc.).

### (2. Evaluation of Relationship Between PSA Level in Serum and AAT/Cre Value in Urine for Prostate Cancer)

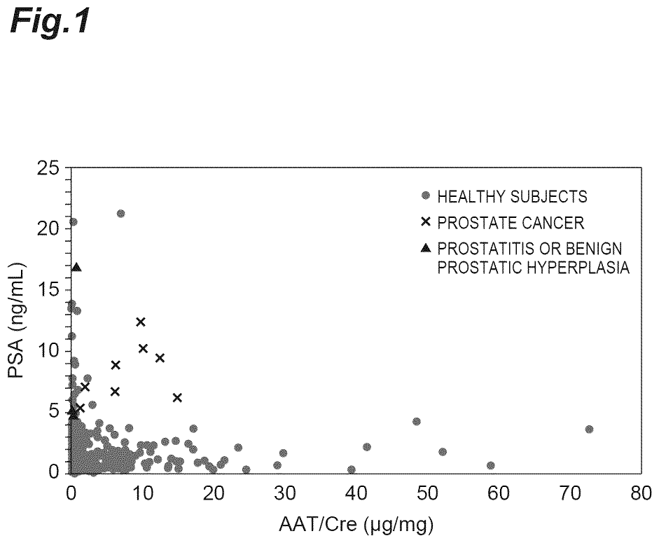

It was confirmed that eight subjects developed prostate cancer within 2 years of their checkup. Additionally, three subjects were diagnosed with prostatitis or benign prostatic hyperplasia. Each of 718 subjects, of which healthy subjects were 707 in total excluding eight subjects confirmed to develop prostate cancer and three subjects with prostatitis or benign prostatic hyperplasia, was evaluated for the relationship between the PSA level in serum and the AAT/Cre value in urine. FIG. 1 is a scatter diagram plotting the PSA levels in serum and the AAT/Cre values in urine.

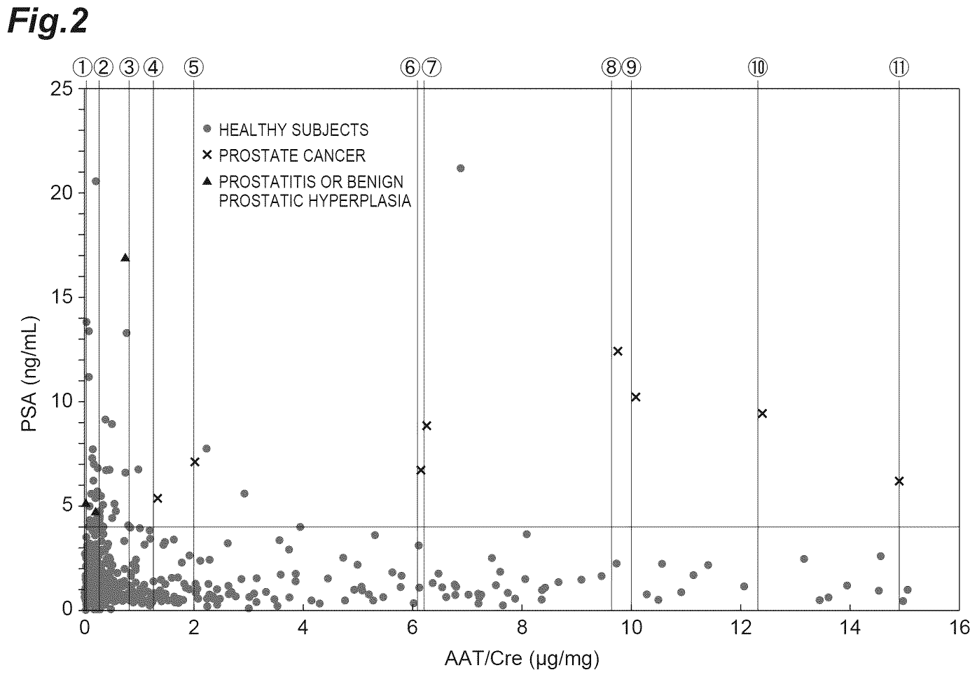

The sensitivity and the specificity were calculated when the threshold value for the PSA level in serum was set to 4.0 ng/ml and the threshold value for the AAT/Cre value in urine was changed. The sensitivity and the specificity were calculated separately for the case in which only the threshold value for the PSA level in serum was set (No. 1), the case in which only the threshold value for the AAT/Cre value in urine was set (Nos. 2, 4, 6, 8, 10, 12, 14, 16, 18, 20, 22, and 24), and the case in which the threshold values for both PSA level in serum and AAT/Cre value in urine were set (Nos. 3, 5, 7, 9, 11, 13, 15, 17, 19, 21, 23, and 25). The results are shown in Table 1 and Table 2. FIG. 2 is a diagram in which auxiliary lines indicating the set threshold value for the PSA level in serum and the set threshold values for the AAT/Cre value in urine were added to the scatter diagram of FIG. 1 and the scale was enlarged. In FIG. 2, a transverse line indicates the threshold value for the PSA level in serum, and vertical lines 1 to 11 indicate the threshold values for the AAT/Cre value in urine. The vertical lines 1 to 11 (threshold values for the AAT/Cre value in urine) respectively correspond to the threshold values for the AAT/Cre value at Nos. 2 and 3, 4 and 5, 6 and 7, 8 and 9, 10 and 11, 12 and 13, 14 and 15, 16 and 17, 18 and 19, 20 and 21, and 22 and 23.

In addition, when analysis based on a receiver operating characteristic (ROC) curve of the AAT/Cre value in urine was performed, a threshold value (Youden Index) at which (sensitivity+specificity−1) reached a maximum value was 1.3 μg/mg.

TABLE 1

| | No. | No. | No. | No. | No. | No. | No. | No. | No. | No. | No. | No. | No. |
|---|---|---|---|---|---|---|---|---|---|---|---|---|---|
| | 1 | 2 | 3 | 4 | 5 | 6 | 7 | 8 | 9 | 10 | 11 | 12 | 13 |
| Threshold value for PSA level | 4.0 | — | 4.0 | — | 4.0 | — | 4.0 | — | 4.0 | — | 4.0 | — | 4.0 |
| in serum (ng/ml) | | | | | | | | | | | | | |
| Threshold value for AAT/Cre | — | 0.03 | 0.03 | 0.2 | 0.2 | 0.8 | 0.8 | 1.3 | 1.3 | 2.0 | 2.0 | 6.2 | 6.2 |
| value in urine (μg/mg) | | | | | | | | | | | | | |
| Sensitivity | 1 | 1 | 1 | 1 | 1 | 1 | 1 | 1 | 1 | 0.875 | 0.875 | 0.75 | 0.75 |
| Specificity | 0.935 | 0.024 | 0.937 | 0.414 | 0.956 | 0.701 | 0.986 | 0.782 | 0.993 | 0.827 | 0.993 | 0.911 | 0.997 |

TABLE 2

| | No. | No. | No. | No. | No. | No. | No. | No. | No. | No. | No. | No. |
|---|---|---|---|---|---|---|---|---|---|---|---|---|
| | 14 | 15 | 16 | 17 | 18 | 19 | 20 | 21 | 22 | 23 | 24 | 25 |
| Threshold value for PSA | — | 4.0 | — | 4.0 | — | 4.0 | — | 4.0 | — | 4.0 | — | 4.0 |
| level in serum (ng/ml) | | | | | | | | | | | | |
| Threshold value for | 6.3 | 6.3 | 9.8 | 9.8 | 10.1 | 10.1 | 12.4 | 12.4 | 14.9 | 14.9 | >14.9 | >14.9 |
| AAT/Cre value in urine | | | | | | | | | | | | |
| (μg/mg) | | | | | | | | | | | | |
| Sensitivity | 0.625 | 0.625 | 0.5 | 0.5 | 0.375 | 0.375 | 0.25 | 0.25 | 0.125 | 0.125 | 0 | 0 |
| Specificity | 0.911 | 0.997 | 0.951 | 0.999 | 0.951 | 0.999 | 0.961 | 0.999 | 0.969 | 0.999 | 0.969 | 0.999 |

When the threshold value for the PSA level in serum was set to 4.0 ng/ml as the cutoff value for PSA test values (No. 1), the sensitivity was 1 (100%) and the specificity was 0.935 (93.5%), and there were a total of 46 examinees who were so-called false positives.

When the threshold value for the AAT/Cre value in urine was set to 1.3 μg/mg as the optimum cutoff value (No. 8), the sensitivity was 1 (100%) and the specificity was 0.782 (78.2%), and there were a total of 155 examinees who were so-called false positives.

Furthermore, as shown in Tables 1 and 2, when the threshold value for the AAT/Cre value in urine and the threshold value for the PSA level in serum were combined and the subjects were separated (Nos. 3, 5, 7, 9, 11, 13, 15, 17, 19, 21, 23, and 25), it became clear that the specificity increased as the threshold value for the AAT/Cre value in urine increased. This means that, when the threshold value for the PSA level in serum was set to 4.0 ng/ml as the cutoff value of the PSA test value, false positive subjects in the subjects having the PSA level in serum equal to or more than the above threshold value can be reduced by further combining the comparison with the threshold value for the AAT/Cre value in urine. In particular, in a case in which the threshold value for the AAT/Cre value in urine was set to 1.3 μg/mg, when the subjects were separated by combining with the setting of the threshold value (4.0 ng/ml) for the PSA level in serum, the specificity was about 0.993 (99.3%) while maintaining the sensitivity to 1 (100%), and there were a total of five subjects who were so-called false positives.

On the other hand, when the threshold value for the AAT/Cre value in urine was set to 2.0 μg/mg, the sensitivity was 0.875 (87.5%) although the specificity did not change. When the threshold value for the PSA level in serum was set to 4.0 ng/ml as the cutoff value of the PSA test value, all of the eight subjects confirmed to develop prostate cancer among the subjects having the PSA level in serum equal to or more than the above threshold value cannot be determined to be positive, meaning that there were so-called false negative subjects. Furthermore, from the results in Tables 1 and 2, it was clarified that as the threshold value for the AAT/Cre value in urine was set higher, the specificity increased more and conversely the sensitivity decreased more.

As can be understood from FIG. 2, the subjects diagnosed with prostatitis or benign prostatic hyperplasia also tended to have a high PSA level in serum. Accordingly, in a conventional method of comparing the PSA level in serum of a subject with a threshold value, it was difficult to determine whether the subject has prostatitis or benign prostatic hyperplasia or has prostate cancer. On the other hand, according to the present invention, as shown in FIG. 2, by appropriately setting the first and second threshold values (in the case of the present Examples, for example, the threshold value for the PSA level in serum was set to 4 ng/mL, and the threshold value for the AAT/Cre value in urine was set to 0.8 μg/mg), subjects with prostate cancer and subjects with prostatitis or benign prostatic hyperplasia can be distinguished from each other, and it was clarified that the specificity of determining prostate cancer increased.

From the above results, when subjects in which the PSA level in serum is equal to or more than an arbitrary threshold value are separated by combining with the AAT/Cre value in urine, it is possible to reduce false positive subjects regarded as a problem in the examination alone based on the quantitative measurement of the PSA level in serum and the comparison with the threshold value. Regarding the threshold value for the AAT/Cre value in urine, the threshold value can also be set according to purposes such as whether to emphasize a high level of a positive rate (high sensitivity) obtained from a quantitative value of the PSA level in serum, whether to emphasize a low level of a false positive rate (high specificity), and to what extent a positive rate and a false positive rate are balanced.

What is claimed is:

1. A method for determining whether a subject has developed or is suspected to have developed prostate cancer, the method comprising:
comparing a prostate-specific antigen (PSA) level in serum with a first threshold value;
comparing an α1-antitrypsin (AAT) level in urine with a second threshold value; and
determining whether a subject in which the PSA level in serum is the first threshold value or more and the AAT level in urine is the second threshold value or more has developed or is suspected to have developed prostate cancer.

2. The method according to claim 1, wherein the AAT level in urine is a level standardized with an internal standard substance.

3. The method according to claim 2, wherein the internal standard substance is creatinine (Cre).

4. The method according to claim 3, wherein the AAT level in urine is a level standardized by dividing the AAT level in urine by a Cre level in urine.

5. The method according to claim 4, wherein the second threshold value is in a range of equal to or more than 0.01 μg/mg to less than 2 μg/mg.

6. The method according to any one of claim 1, wherein the first threshold value is in a range of equal to or more than 1 ng/mL and equal to or less than 5 ng/mL.

7. A screening method for selecting a subject that has developed or is suspected to have developed prostate cancer, the method comprising:
comparing a prostate-specific antigen (PSA) level in serum with a first threshold value;
comparing an α1-antitrypsin (AAT) level in urine with a second threshold value; and
selecting a subject in which the PSA level in serum is the first threshold value or more and the AAT level in urine is the second threshold value or more as a subject that has developed or is suspected to have developed prostate cancer.

8. The method according to claim 7, wherein the AAT level in urine is a level standardized with an internal standard substance.

9. The method according to claim 8, wherein the internal standard substance is creatinine (Cre).

10. The method according to claim 9, wherein the AAT level in urine is a level standardized by dividing the AAT level in urine by a Cre level in urine.

11. The method according to claim 10, wherein the second threshold value is in a range of equal to or more than 0.01 μg/mg to less than 2 μg/mg.

12. The method according to any one of claim 7, wherein the first threshold value is in a range of equal to or more than 1 ng/ml and equal to or less than 5 ng/mL.

13. A data collection method for determining whether a subject has developed or is suspected to have developed prostate cancer, the method comprising:
comparing a prostate-specific antigen (PSA) level in serum with a first threshold value; and
comparing an α1-antitrypsin (AAT) level in urine with a second threshold value.

14. The method according to claim 13, wherein the AAT level in urine is a level standardized with an internal standard substance.

15. The method according to claim 14, wherein the internal standard substance is creatinine (Cre).

16. The method according to claim 15, wherein the AAT level in urine is a level standardized by dividing the AAT level in urine by a Cre level in urine.

17. The method according to claim 16, wherein the second threshold value is in a range of equal to or more than 0.01 μg/mg to less than 2 μg/mg.

18. The method according to any one of claim 13, wherein the first threshold value is in a range of equal to or more than 1 ng/ml and equal to or less than 5 ng/mL.
